(19) United States
(12) Patent Application Publication
(10) Pub. No.: US 2013/0085376 A1
(43) Pub. Date: Apr. 4, 2013

(54) MEDICAL IMAGING METHOD AND A SYSTEM TO SYNCHRONIZE A BREATHING COMMAND WITH THE PATIENT'S BREATHING CYCLE

(71) Applicant: Martin Ringholz, Erlangen (DE)

(72) Inventor: Martin Ringholz, Erlangen (DE)

(21) Appl. No.: 13/627,305

(22) Filed: Sep. 26, 2012

(30) Foreign Application Priority Data
Sep. 26, 2011 (DE) 102011083387.0

(51) Int. Cl. A61B 5/055; A61B 5/00

(52) U.S. Cl. CPC A61B 5/055; A61B 5/741
USPC 600/413

(57) ABSTRACT

In a medical imaging method to synchronize a breathing command with a breathing cycle of a patient breathing parameters of a breathing cycle of the patient are detected, a reconstructed breathing cycle of the patient is reconstructed using the detected breathing parameters, a breathing command is generated in coordination with a medical imaging measurement, and the breathing command is automatically emitted as an output to the patient.

### BACKGROUND OF THE INVENTION

1. Field of the Invention

The invention proceeds from a medical imaging method and system to synchronize a breathing command with a breathing cycle of a patient.

2. Description of the Prior Art

In medical imaging examinations, for example, in magnetic resonance examinations and/or computed tomography examinations, it is typical (particularly in examinations in the abdominal region and/or in the cardiac region of the patient) for breathing commands to be given to the patient before medical image exposures (for example “inhale”, “exhale”, “don't inhale anymore now,” etc.). These breathing commands are transmitted from clinical personnel in charge of the medical imaging method to the patient by means of a speaker system. Depending on the breathing phase of the patient, this can lead to problems in the breathing of the patient. For example, at the point in time of the breathing command the patient may be in a phase opposite said breathing command, such that the patient must quickly change or adapt his or her breathing.

For example, if image data acquisitions are implemented repeatedly and/or if longer acquisitions are implemented, this can lead to shortness of breath and/or gasping on the part of the patient, and may result in the patient can no longer being able to adapt his or her breathing to the image acquisition or the breathing commands, at least in part. The exposures thus cannot be used for a meaningful analysis, such that the acquisitions must be implemented again.

### SUMMARY OF THE INVENTION

An object of the present invention is to provide a medical imaging method in which a breathing command to the patient for a medical imaging measurement takes place with adaptation to the breathing cycle of the patient.

In accordance with the invention a medical imaging method to synchronize the breathing command with a breathing cycle of a patient includes the following steps:
detect breathing parameters of the breathing cycle of the patient,
reconstruct a reconstructed breathing cycle of the patient using the detected breathing parameters,
in a processor, generate a breathing command in coordination with a medical imaging measurement and
automatically emit the breathing command to the patient.

The generation of the breathing command according to the invention is adapted to the breathing cycle of the patient, and a breathing command that is contrary to a current breathing phase of the patient thus can advantageously be prevented. Medical imaging measurements can also be designed to be more comfortable for the patient, such that breathing difficulties of the patient during the medical imaging measurement can be reduced and/or prevented. The medical imaging measurement thus can be concluded successfully, and time-intensive or cost-intensive repeat measurements can additionally advantageously be prevented. Due to the avoidance of repeat measurements, for example, the patient can be exposed to a lower radiation dose. This is particularly advantageous in medical imaging measurements with a computed tomography device so that such the risk to the patient can be minimized. The medical imaging measurement is advantageously a magnetic resonance measurement and/or a computed tomography measurement and/or additional measurements that appear to be reasonable to those skilled in the art. In this context by a “breathing parameter” means a parameter that at least partially characterizes the breathing cycle of the patient, for example an inhalation point in time and/or an exhalation point in time.

The automated output of the breathing command preferably takes place after an input of a manual start signal. For example, the manual start signal is entered and/or triggered by a technician in charge of the medical imaging measurement. The breathing command can be selected correctly, and in addition to this the start signal can be adapted to the medical imaging measurement, in particular to acquisitions with a specific breathing position of the patient.

Furthermore, the automated output of the breathing command takes place depending on an average breathing cycle. The breathing command can be particularly effectively adapted to a current breathing cycle of the patient. For example, if the manual triggering of the start signal for the output of the breathing command takes place at a point in time at which the breathing command is contrary to a current breathing phase of the patient, the automated output of the breathing command can be interrupted until there is an advantageous agreement and/or match of the breathing command with the current breathing phase of the patient. An average breathing cycle means a reconstructed breathing cycle that essentially calculates a mean value from multiple breathing cycles of the patient.

If an averaging of at least two breathing cycles of the patient is taken into account for a reconstruction of the average breathing cycle of the patient, an adaptation of the output of breathing commands that is optimized to the patient in terms of time can be achieved. The breathing cycles that are considered for the reconstruction are advantageously formed by breathing cycles of the patient that said patient executes immediately before the reconstruction. An averaging of at least three breathing cycles of the patient, and preferably of five breathing cycles of the patient, is advantageously used for the reconstruction.

The invention also encompasses from a medical imaging system to execute a medical imaging method as described above, with a detector unit to implement a medical imaging measurement, a breathing detection unit to detect breathing parameters of a patient, an evaluation unit, and an output unit.

The evaluation unit is designed to reconstruct a reconstructed breathing cycle using the detected breathing parameters, and to generate a breathing command for the patient depending on this reconstructed breathing cycle, and to output this breathing command to the patient by means of the output unit. An advantageous matching between the breathing cycle of the patient and an output of the breathing command can take place in that the output of the breathing command can hereby be adapted to the breathing cycle of the patient. For example, the breathing detection unit can be formed by a chest breathing belt and/or additional breathing detection units that appear to be reasonable to those skilled in the art.

An effective adaptation of the output of the breathing command to the breathing cycle of the patient can be achieved if the breathing command is formed at least in part by an automated speech output. In particular, delays in the output of the breathing command and/or an output of the breathing command that is too early can hereby be prevented.

Furthermore, the medical imaging system in accordance with the invention has an input unit by means of which a start signal for the automated speech output can be manually entered. The breathing command can thereby be selected correctly, and in addition the start signal can be adapted to the medical imaging device. The input device can be formed by a keyboard and/or a computer mouse and/or additional input units.

In a further embodiment of the invention, the evaluation unit reconstructs an average breathing cycle for an output of the breathing command that is adapted to the reconstructed breathing cycle. The breathing command can be particularly effectively adapted to a current breathing cycle of the patient.

### BRIEF DESCRIPTION OF THE DRAWINGS

FIG. 1 is a flowchart showing the basic step of the method according to the invention.

FIG. 2 schematically illustrates a medical imaging system according to the invention.

### DESCRIPTION OF THE PREFERRED EMBODIMENTS

A medical imaging system 10 according to the invention is shown in FIG. 2, In this exemplary embodiment, the medical imaging system 10 is formed by a magnetic resonance device, as an example. As an alternative, the medical imaging system 10 can be formed by a computed tomography device and/or a PET device, etc.

The magnetic resonance device has a data acquisition unit formed by a magnet unit 11, with a basic magnet 12 to generate a strong (and constant) basic magnetic field 13. The magnetic resonance device additionally has a cylindrical acquire region 14 to accommodate a patient 15. The acquisition region 14 is cylindrically surrounded by the magnet unit 11 in the circumferential direction. The patient 15 can be moved into the acquisition region 14 by a patient bed 16 of the magnetic resonance device. For this purpose, the patient bed 16 is movable within the acquisition region 14.

Furthermore, the magnet unit 11 has a gradient coil 17 to generate a magnetic field gradient that is used for a spatial coding during an imaging. The gradient coil 17 is controlled by a gradient control unit 18. The magnet unit 11 furthermore has a radio-frequency antenna unit that has a radio-frequency antenna 19 and a radio-frequency antenna control unit 20. An alignment of the nuclear spins in the direction of the basic magnetic field 13 generated by the basic magnet 12 occurs. The radio-frequency antenna 19 is controlled by the radio-frequency antenna control unit 20 and radiates radio-frequency magnetic resonance sequences into an examination space that is essentially formed by the acquisition region 14 that deflect the nuclear spins from the aforementioned alignment, and as they precess as a result of this excitation, the emit magnetic resonance signals that are received (detected) by the radio-frequency antenna unit.

The magnetic resonance device has a control unit 21 formed by a computer to control the basic magnet 12, the gradient coil unit 18 and the radio-frequency antenna control unit 20. The control unit 21 centrally controls the magnetic resonance device, for example the implementation of a predetermined imaging gradient echo sequence. Control information—for example imaging parameters—as well as reconstructed magnetic resonance images can be displayed at a display unit 22 (for example on at least one monitor) of the magnetic resonance device for an operator of said magnetic resonance device. In addition, the magnetic resonance device has an input unit 23 that allows information and/or parameters to be entered by an operator during a measurement process.

Furthermore, the magnetic resonance device has a breathing detection unit 24 with which breathing parameters of a breathing cycle of the patient 15 are detected for a magnetic resonance examination. In the exemplary embodiment, the breathing detection unit 24 is formed by a chest breathing belt. Further embodiments of the breathing detection unit 24 that appear to be reasonable to those skilled in the art may additionally be present. The data detected by the breathing detection unit 24 are relayed via a data line (not shown) to an evaluation unit 25 and evaluated there. A reconstructed breathing cycle of the patient 15 is reconstructed and/or calculated using the detected breathing parameters. A medical imaging method to synchronize a breathing command is additionally controlled by means of the evaluation unit 25. For this purpose, the evaluation unit 25 has a processor and has the computer programs and software required for a control and reconstruction, which computer programs and software are stored in a memory unit (not shown in detail) of the evaluation unit 25. In addition, the evaluation unit 25 can also be formed by the control unit 21.

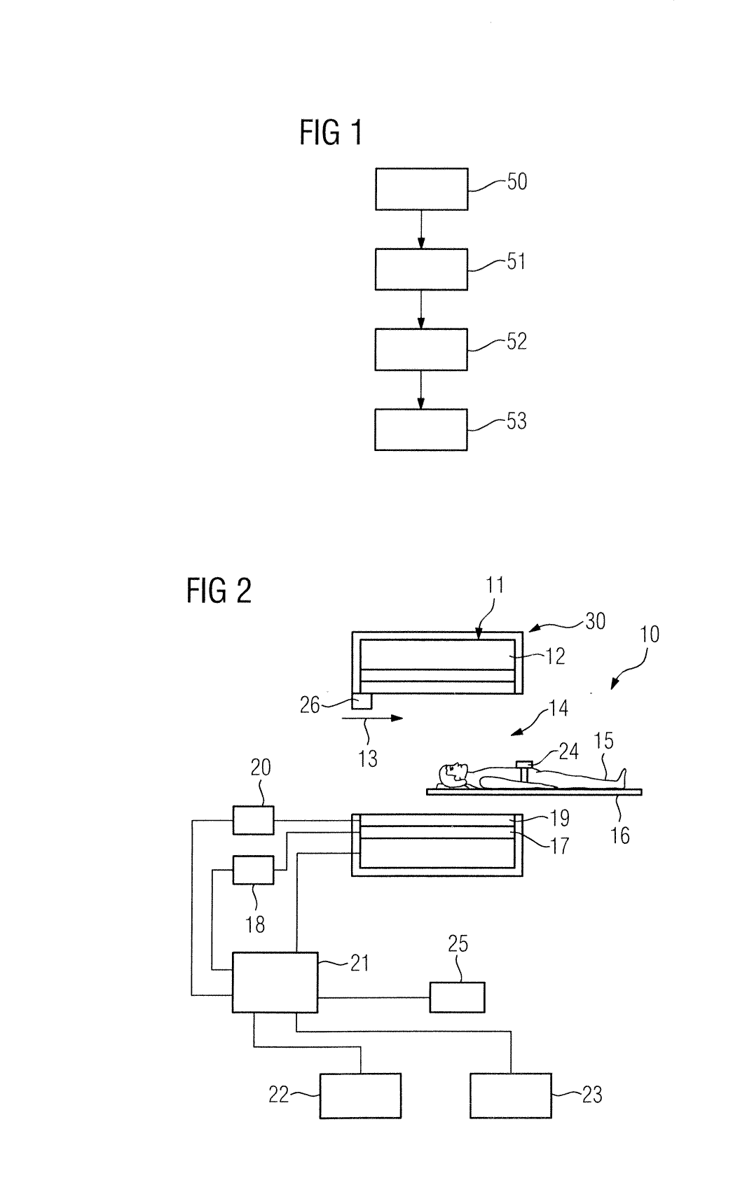

The medical imaging method for synchronization of a breathing command of a patient 15 is presented in detail in the following (FIG. 1). First, the patient 15 is positioned on the patient bed 16 and the breathing detection unit 24 is positioned around the patient 15 such that said breathing detection unit 24 can detect parameters (in particular breathing parameters) from which a reconstructed breathing cycle of the patient 15 can be calculated. The patient bed 16 with the patient 15 thereupon is driven into the acquisition region 14 of the magnetic resonance device. Finally, the medical imaging method is started to synchronize the breathing command. Breathing parameters of the patient 15 are hereby detected by the breathing detection unit 24 in a detection step 50. The data acquired by the breathing detection unit 24 are subsequently relayed to the evaluation unit 24 and evaluated there in a reconstruction step 51. A reconstructed breathing cycle of the patient 15 is reconstructed in the evaluation unit 25 using the detected breathing parameters.

A breathing command is subsequently generated by the evaluation unit 25 in a generation step 52, wherein the breathing command takes place in coordination with a medical imaging measurement (in particular a magnetic resonance measurement). For example, the breathing command can be one of the commands “please inhale”, “please exhale”, “please stop your breathing,” etc. The evaluation unit 25 generates the breathing command as an acoustic speech command that is output via an output unit 26 (formed by a loudspeaker unit) of the magnetic resonance device.

As soon as the breathing command is provided or has been generated, this is communicated by the evaluation unit 25, via the display device 22, to the clinical personnel in charge of the magnetic resonance device. In an output step 53, a manual start signal is subsequently input by the clinical personnel via the input unit 23 and relayed to the evaluation unit 25. As soon as the manual start signal is present, the breathing command is output by the evaluation unit 25 in the output step 53 by an automated output. However, the automated output of the breathing command is adapted by the evaluation unit 25 to the current breathing cycle of the patient 15, or the automated output of the breathing command is synchronized with the current breathing cycle of the patient 15. For example, if the patient 15 is presently in an inhalation phase of the breathing cycle, no breathing command that requires the patient 15 to inhale is output by the evaluation unit 25. In contrast, the evaluation unit 25 waits with the output of the breathing command (which orders the patient 15 to inhale, for example) until the breathing cycle of the patient 15 has reached a phase in which said patient 15 can execute the breathing command immediately after its output, without thereby needing to interrupt his or her current breathing cycle and executing a breathing phase that is contrary to the current phase of the breathing cycle.

For this purpose, the evaluation unit 25, together with the breathing detection unit 24, determines multiple breathing cycles of the patient 15 before the automated output of the breathing command, and from these calculates an average breathing cycle so that fluctuations in the breathing of the patient 15 can be kept as small as possible for the reconstruction of the breathing cycle. A number of breathing cycles that are detected for the reconstruction of the average breathing cycle of the patient 15 thereby amounts to at least two breathing cycles, preferably three breathing cycles and more preferably five breathing cycles.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical imaging method comprising:
detecting breathing parameters of a breathing cycle of a patient with a breathing parameter detection device;
supplying said breathing parameters from said breathing parameter detection device to a processor and, in said processor, automatically reconstructing a reconstructed breathing cycle of the patient using the detected breathing parameters;
in said processor, generating a breathing command coordinated with a medical imaging measurement to be performed on the patient, based on the reconstructed breathing cycle; and
automatically emitting said breathing command in humanly perceptible form to the patient as an output from said processor and operating a medical imaging system in coordination with said breathing command to acquire a medical image of the patient.

2. A medical imaging method as claimed in claim 1 comprising automatically emitting said breathing command from said processor after manually entering a start signal into said processor.

3. A medical imaging method as claimed in claim 1 comprising emitting said output of said breathing command from said processor based on an average breathing cycle, as said reconstructed breathing cycle.

4. A method as claimed in claim 3 comprising generating said average breathing cycle as an average of at least two breathing cycles of the patient, represented by said breathing parameters.

5. A medical imaging system comprising:
a breathing parameter detection device configured to detect breathing parameters of a breathing cycle of a patient with;
a processor supplied with said breathing parameters from said breathing parameter detection device, said processor being configured to automatically reconstruct a reconstructed breathing cycle of the patient using the detected breathing parameters;
said processor being configured to generate a breathing command coordinated with a medical imaging measurement to be performed on the patient, based on the reconstructed breathing cycle; and
said processor being configured to automatically emit said breathing command in humanly perceptible form to the patient as an output from said processor.

6. A medical imaging system as claimed in claim 5 wherein said processor is configured to automatically generating a human speech command as said output.

7. A medical imaging system as claimed in claim 6 wherein said processor comprises an input unit configured to allow manual input of a start signal to said processor for emitting said automated speech command.

8. A medical imaging system as claimed in claim 5 wherein said processor is configured to generate an average breathing cycle of the patient, as said reconstructed breathing cycle, from said breathing parameters.
